(19) United States
(12) Patent Application Publication
(10) Pub. No.: US 2015/0250449 A1
(43) Pub. Date: Sep. 10, 2015

(54) PUNCTURE ADAPTER AND ULTRASOUND PROBE

(71) Applicants: Kabushiki Kaisha Toshiba, Minato-ku (JP); Toshiba Medical Systems Corporation, Otawara-shi (JP)

(72) Inventors: Kentaro TSUZUKI, Nasushiobara (JP); Takashi TAKEUCHI, Otawara (JP); Hideki KOSAKU, Nasushiobara (JP); Hiroyuki SHIKATA, Nasushiobara (JP)

(73) Assignees: Kabushiki Kaisha Toshiba, Minato-ku (JP); Toshiba Medical Systems Corporation, Otawara-shi (JP)

(21) Appl. No.: 14/720,462

(22) Filed: May 22, 2015

Related U.S. Application Data
(63) Continuation of application No. PCT/JP2013/081420, filed on Nov. 21, 2013.

(30) Foreign Application Priority Data
Nov. 23, 2012 (JP) 2012-256977

(51) Int. Cl. A61B 8/00; A61B 8/12

(52) U.S. Cl. CPC A61B 8/445; A61B 8/12

(57) ABSTRACT

A puncture adapter includes a main body, a pair of operation parts, a pair of engaging parts, and a pair of connectors. The main body includes a first part having a first width and a second part having a second width that is narrower than the first width. The operation parts come close to each other in response to the removal operation. Each of the engaging parts is integrally formed with either one of the operation parts, and is engageable with an ultrasound probe. Each of the connectors connects one end in the width direction of the second part and a position between one of the operation parts and one of the engaging parts. When the operation parts come close to each other in response to the removal operation, the engaging parts separate from each other to release the engagement.

### CROSS-REFERENCE TO RELATED APPLICATION

This application is based upon and claims the benefit of priority from Japanese Patent Application No. 2012-256977, filed 23 Nov. 2012; the entire contents of which are incorporated herein by reference.

### FIELD

Embodiments described herein relate generally to a puncture adapter and an ultrasound probe.

### BACKGROUND

Ultrasound diagnosis is a diagnostic approach in which a subject is scanned with ultrasound waves for imaging the biological information based on a received signal generated from reflected waves of the ultrasound waves. The ultrasound waves are transmitted and received through an ultrasound probe.

An ultrasound probe that is provided with various instruments enables more advanced diagnosis and treatment. Examples of such diagnosis and treatment include a puncture to correct tissues and fluids, discharge fluids and pus, or infuse a drug by inserting a puncture needle in the body. In a puncture, the ultrasound probe is fitted with an instrument (puncture adapter) to guide a puncture needle. In a puncture performed by inserting an ultrasound probe in the body, with the use of an ultrasound probe that has a shape corresponding to the insertion site and a puncture adapter having a shape along with the ultrasound probe, the insertability and operability are attempted to be improved.

The puncture adapter is provided with a functional part related to the removal operation from the ultrasound probe. This functional part includes a pair of portions (operation parts) that is gripped between two fingers and a connector that connects the main body of the adapter for holding the puncture needle and each operation part.

For an ultrasound probe to be inserted in the body, it is desirable that the puncture adapter have a small shape, and in particular, a width be reduced in a direction perpendicular to the insertion direction to prevent degradation of operability and an interference with a patient.

However, if the puncture adapter is designed to be small, it becomes difficult to secure the length of the connector. This increases the rigidity of the connector. Accordingly, a larger operating force (a force to grip the pair of the operation parts) is required to function the functional part, making the removal difficult. On the other hand, if the connector is designed to be long to reduce the operating force, the size of the puncture adapter is increased.

### BRIEF DESCRIPTION OF THE DRAWINGS

FIG. 1 is a schematic diagram of a configuration example according to an embodiment;

FIG. 2 is a schematic diagram of a configuration example according to an embodiment;

FIG. 3 is a schematic diagram of a configuration example according to an embodiment;

FIG. 4 is a schematic diagram of a configuration example according to an embodiment;

FIG. 5 is a schematic diagram of a configuration example according to an embodiment;

FIG. 6 is a schematic diagram of a configuration example according to an embodiment;

FIG. 7 is a schematic diagram of a configuration example according to an embodiment;

FIG. 8A is a schematic diagram of a configuration example according to an embodiment; and

FIG. 8B is a schematic diagram of a configuration example according to an embodiment.

### DETAILED DESCRIPTION

In general, according to one embodiment, a puncture adapter is configured to hold a puncture needle and be attached to an ultrasound probe that is configured to be inserted in a subject. The puncture adapter includes a main body, a pair of operation parts, a pair of engaging parts, and a pair of connectors. The main body includes a first part having a first width and a second part having a second width that is narrower than the first width. The operation parts come close to each other in response to operation for removing the puncture adapter from the ultrasound probe. Each of the engaging parts is integrally formed with either one of the operation parts, and is engageable with the ultrasound probe. One of the connectors connects one end in the width direction of the second part and a position between one of the operation parts and one of the engaging parts. The other of the connectors connects the other end in the width direction of the second part and a position between the other of the operation parts and the other of the engaging parts. When the operation parts come close to each other in response to the operation, the engaging parts separate from each other to release the engagement.

In the following, a description is given of an example of a puncture adapter and an ultrasound probe according to an embodiment with reference to the drawings. The puncture adapter of the embodiment is attachable/detachable to/from the ultrasound probe that is insertable in a subject. The ultrasound probe of the embodiment includes the puncture adapter of the embodiment and an ultrasound probe (ultrasound probe body, i.e., the main body of the ultrasound probe) that is insertable in a subject.

### [Configuration of the Ultrasound Probe]

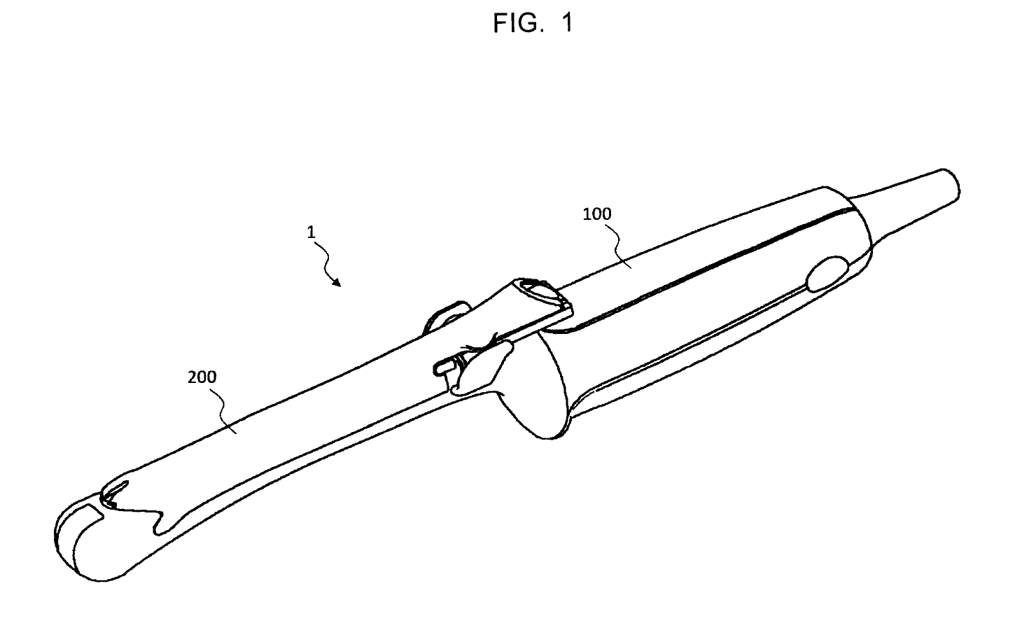

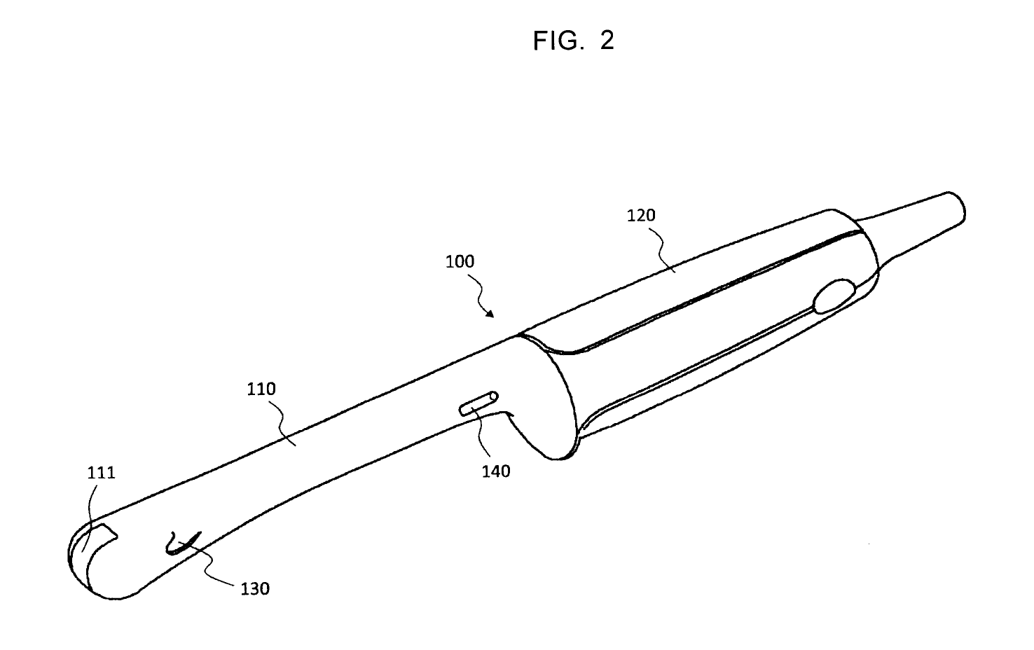

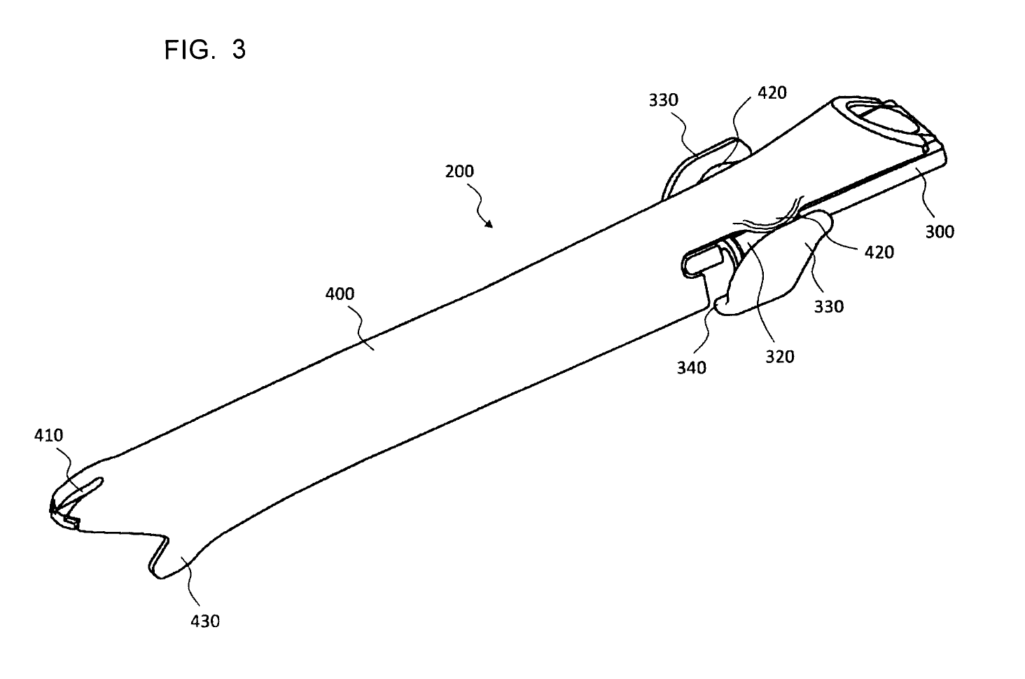

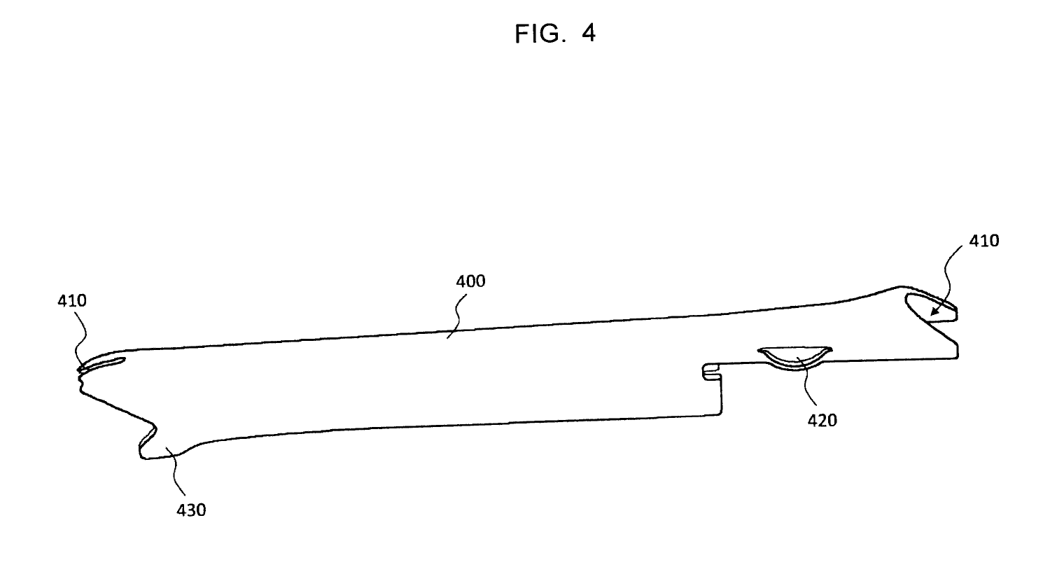

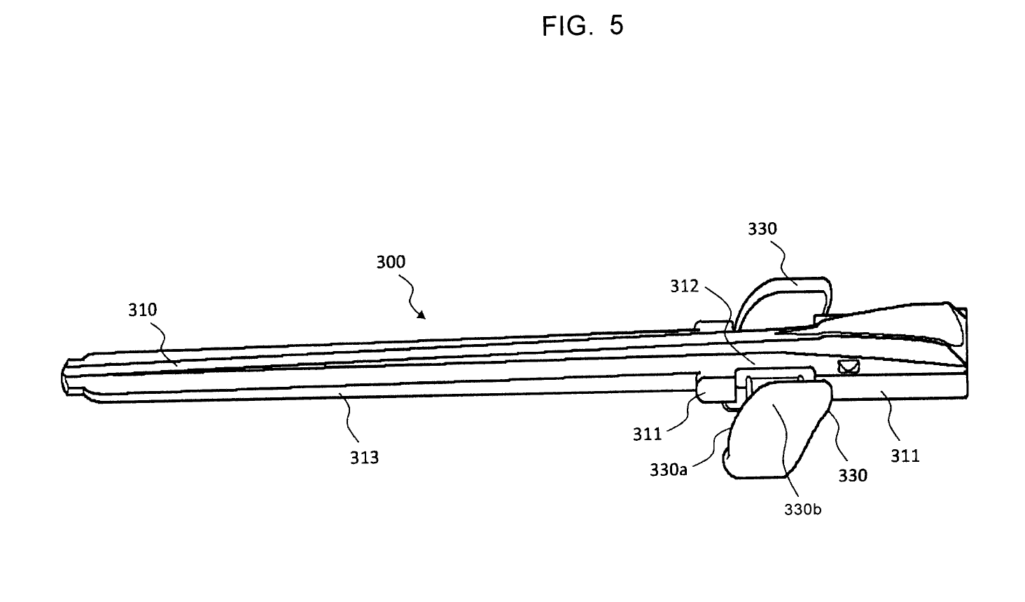

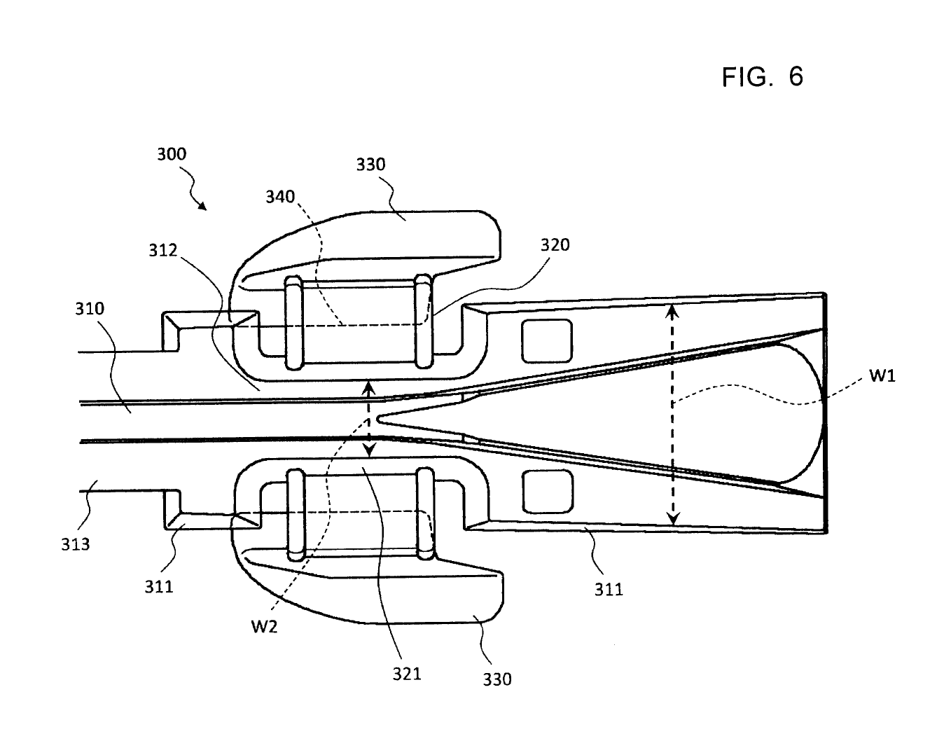

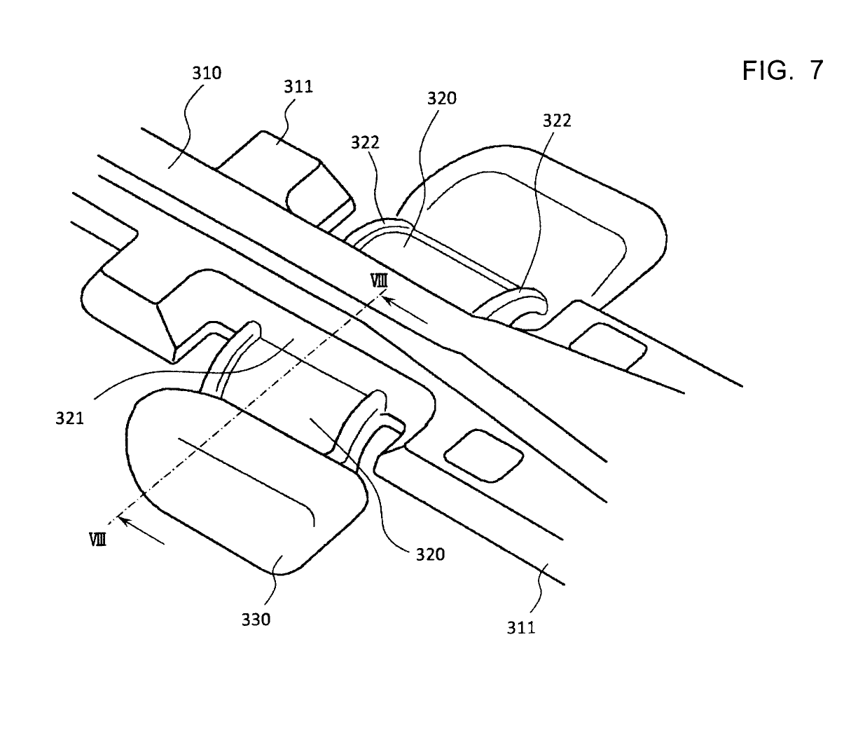

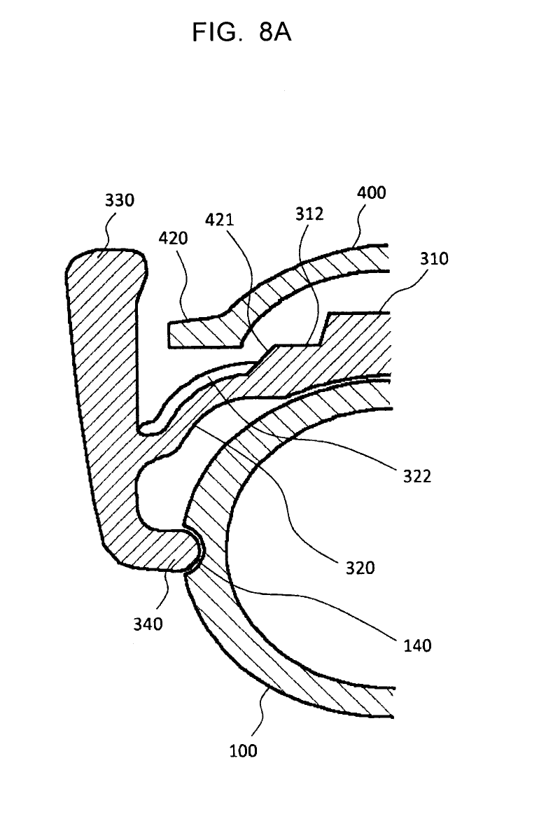

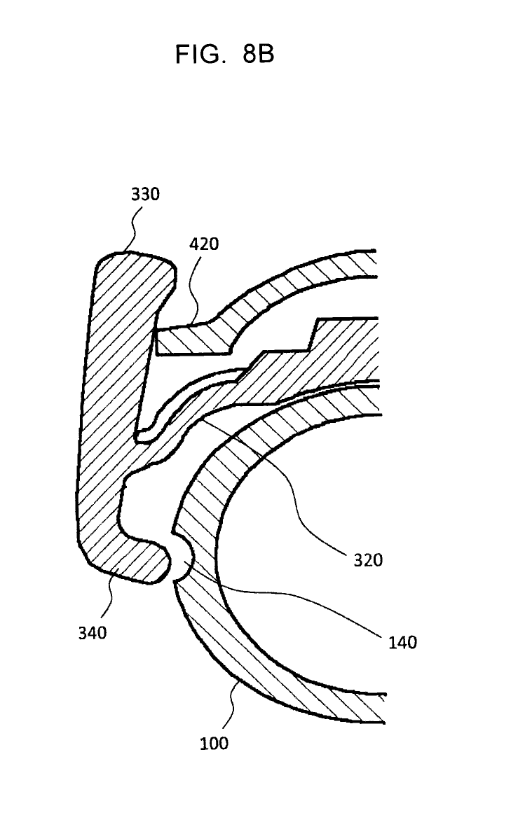

Described below is the configuration of the ultrasound probe of the embodiment. FIGS. 1 to 8B each illustrate an example of the configuration of the ultrasound probe. FIG. 1 is a perspective view of the ultrasound probe fitted with the puncture adapter. FIG. 2 is a perspective view of the ultrasound probe from which the puncture adapter is removed. FIG. 3 is a perspective view of the puncture adapter. FIG. 4 is a perspective view of a part (cover) of the puncture adapter. FIG. 5 is a perspective view of a part of the puncture adapter (adapter body, i.e., the main body of the adapter). FIG. 6 is a top view of a part of the puncture adapter. FIG. 7 is a perspective view of a part of the puncture adapter. FIGS. 8A and 8B are cross-sectional views, taken along line VIII-VIII of FIG. 7, illustrating the operation of the puncture adapter. An ultrasound probe 1 illustrated in FIG. 1 includes an ultrasound probe body 100 and a puncture adapter 200.

### (Ultrasound Probe Body 100)

As illustrated in FIG. 2, the ultrasound probe body 100 includes an insertion part 110 to be inserted into a subject, and a handle 120 that the user grips. The distal end of the insertion part 110 accommodates a plurality of ultrasound transducers. The ultrasound probe body 100 is a one-dimensional array probe including an array of ultrasound transducers arranged in the scanning direction or a two-dimensional array probe including two dimensional arrays of ultrasound transducers. Ultrasound waves generated by the ultrasound transducers are output from an ultrasound input/output part 111 at the distal end of the insertion part 110. The ultrasound transducers also receive reflected waves from a subject, which have entered through the ultrasound input/output part 111.

Although not illustrated, the handle 120 is connected to a cable for transmitting/receiving signals to/from the main body of an ultrasound diagnostic apparatus. In addition, the handle 120 contains an electrical circuit for processing signals fed from the main body of the ultrasound diagnostic apparatus and the ultrasound transducers.

The ultrasound probe body 100 transmits ultrasound waves to the subject based on a signal received from the main body of the ultrasound diagnostic apparatus. The ultrasound probe body 100 converts the reflected waves from the subject into an electrical signal (echo signal) by the ultrasound transducers, and sends the electrical signal to the main body of the ultrasound diagnostic apparatus. The main body of the ultrasound diagnostic apparatus visualizes the biological information (morphological information, functional information, etc.) of the subject into an image based on the electrical signal received from the ultrasound probe body 100. Such electrical processing and image processing are performed in a conventional manner.

The ultrasound probe body 100 is provided with engaging parts 130 and 140, each on one of the both sides. The engaging parts 130 and 140 are each formed as a recess having a shape corresponding to their engagement counterparts. The engaging part 130 located on the distal end side (distal end side engaging part) engages a distal end side engaging part 430 of the puncture adapter 200 illustrated in FIG. 3 and the like. The engaging part 140 located on the proximal end side (proximal end side engaging part) engages a proximal end side engaging part 340 of the puncture adapter 200 illustrated in FIG. 3 and the like (one or the other of a pair of proximal end side engaging parts. Hereinafter, the proximal end side engaging part 340 refers to each of the pair of proximal end side engaging parts unless specifically otherwise indicated).

### (Puncture Adapter 200)

The puncture adapter 200 has a function of holding a puncture needle (not illustrated) and is configured to be attached to the ultrasound probe body 100 by the engaging parts described above. As illustrated in FIG. 3, the puncture adapter 200 includes an adapter body 300 and a cover 400. The cover 400 is attachable/detachable to/from the adapter body 300.

The puncture needle (not illustrated) is located in a space formed between a puncture needle installation part 310 of the adapter body 300 and a guide groove 410 of the cover 400. The puncture needle is guided in the space along the guide groove 410. The puncture needle installation part 310 and the guide groove 410 correspond to an example of “holder”.

In this embodiment, the adapter body and the cover are formed separately. With this, it is possible to improve the effectiveness and efficiency in cleaning the puncture adapter 200 after use. In particular, it is possible to suitably clean the part (the space, etc.) that the puncture needle makes contact with. Nevertheless, the configuration of the puncture adapter is not to be limited to this. For example, in one embodiment, the puncture adapter may be integrally formed.

### (Adapter Body 300)

As illustrated in FIG. 5 and the like, the adapter body 300 includes a first part 311 having a first width W1 and a second part 312 having a second width W2 that is narrower than the first width W1 (W2<W1). In this embodiment, the first part 311 is provided on both the distal end side and the proximal end side of the second part 312. An insertion part 313 having a shape tapered toward the distal end is provided on the distal end side of the first part 311 that is located on the distal end side.

The first width W1 and/or the second width W2 may be constant or not. In the latter case, the maximum value of the second width W2 is designed to be smaller than the minimum value of the first width W1. Although the difference between the first width W1 and the second width W2 is arbitrary, for example, the second width W2 is designed to be substantially 0.8 times the first width W1.

The adapter body 300 may be integrally formed, or may be formed as a combination of two or more members. The term “connection” and similar terms indicating “connection” as used herein refer not only to the connection between different members, but also intended to refer to the connection between different functional parts in one molded article.

As illustrated in FIGS. 6 and 7, the both ends (both sides) of the second part 312 are each connected to one end (proximal end) of a connector 320 (one or the other of a pair of connectors. Hereinafter, the connector 320 refers to each of the pair of connectors unless specifically otherwise indicated). Here, a space between the connector 320 and the first part 311 may be recognized as a notch (cut). The notch (cut) refers not only to those formed by cutting out a portion corresponding to the space, but it is a concept including a space formed by molding resin or the like and a space formed as a result of combining different members.

On the other end (distal end) side of the connector 320, an operation part 330 (one or the other of the pair of operation parts. Hereinafter, the operation part 330 refers to each of the pair of operation parts unless specifically otherwise indicated) and the proximal end side engaging part 340 are provided. The operation part 330 and the proximal end side engaging part 340 are integrally formed. The end of the connector 320 is connected to a position between the operation part 330 and the proximal end side engaging part 340 with respect to an integral portion including the operation part 330 and the proximal end side engaging part 340.

The connector 320 is formed as a plate-like functional part bent downward from a connection part on the second part 312 side toward a connection part on the operation part 330 side. Here, the term “downward” indicates a direction from the upper surface where the puncture needle installation part 310 is provided toward the lower surface opposite thereto. In other words, the term “downward” indicates a direction from the upper surface to a surface in contact with the ultrasound probe body 100. By providing the connector 320 with such a bent shape, the length of the connector 320 can be sufficiently secured. Note that the length of the connector 320 is a distance between a connection part to the second part 312 and a connection part to the operation part 330 or the like, and indicates a distance measured along the connector 320.

The connector 320, which is a plate-like functional part as described above, is thinner than the second part 312. Accordingly, the rigidity of the connector 320 is low as compared to the second part 312.

As illustrated in FIGS. 7 and 8A, in a connection part (boundary part) between the second part 312 and the connector 320, that is, in the proximal end of the connector 320, a chamfer 321 is formed. As mentioned above, since the second part 312 and the connector 320 have different thicknesses, a step is formed in the connection part between the second part 312 and the connector 320. The chamfer 321 gradually reduces the thickness difference between them as well as avoiding the presence of a corner in the concave direction of this step. The chamfer 321 is provided on at least part of the step between the upper surface of the second part 312 and the upper surface of the connector 320, and is formed to have a surface oriented in a direction different from both of these upper surfaces.

The chamfer of the embodiment may be in any form. For example, the chamfer may be a C-chamfer, the surface of which forms a slope (C-surface cut) or an R-chamfer, the surface of which forms a curve (R-surface cut). Besides, a chamfer may be provided to the step between the lower surface of the second part 312 and the lower surface of the connector 320 or on the side of the connector 320.

As illustrated in FIGS. 7 and 8A, the connector 320 has ribs 322 along its length direction (a direction that connects the connection part on the second part 312 side to the connection part on the operation part 330 side). The ribs 322 are protrusions that protrude upward and extend in the longitudinal direction of the connector 320, and each have a function of increasing the rigidity of the connector 320. The height of the ribs 322 is designed arbitrarily depending on a degree at which the rigidity of the connector 320 is to be increased. Incidentally, as the height of the ribs 322 increases, the rigidity of the connector 320 also increases, and the connector 320 is less likely to be deformed.

In this embodiment, the ribs 322 are provided on the upper surface of the connector 320. When the operation parts 330 are moved in directions coming close to each other in response to operation (described later) to remove the puncture adapter 200 from the ultrasound probe body 100 (see FIGS. 8A and 8B), the connector 320 is deformed in such a manner that the upper surface shrinks. Accordingly, by providing the upper surface with the ribs 322, the rigidity of the connector 320 can effectively improve, and thus the ribs 322 are less likely to be broken due to the deformation of the connector 320. The ribs may be provided on the lower surface of the connector 320.

In this embodiment, the connector 320 is provided with the ribs 322 on both the ends in the width direction to stabilize the deformation of the connector 320. That is, even when stress is applied unevenly to one side in the width direction of the connector 320, the distortion in the width direction of the connector 320 is suppressed by the ribs 322 at both ends. Incidentally, the positions of the ribs 322 are not limited to this example.

The operation part 330 is displaced inward in response to the operation to remove the puncture adapter 200 from the ultrasound probe body 100. This operation is knob operation to move the pair of the operation parts 330 in directions coming close to each other.

As illustrated in FIG. 5, the operation part 330 of this embodiment has a surface 330a on the adapter distal end side. The surface 330a is formed in a convex shape in the adapter distal end direction and also has a smooth shape. Thus, even if the operation part 330 makes contact (interferes) with a subject, this is less likely to damage the subject.

The operation part 330 of this embodiment also has a surface 330b on the adapter proximal end side. The surface 330b is formed in a shape that is inclined to the adapter proximal end side from the connection part to the connector 320 toward the distal end of the operation part 330 (upward). This makes it possible to increase the distance between the fulcrum and the effort to which a user force is applied with respect to the operation part 330. Thereby, the force required for the operation can be reduced. In addition, it is possible to increase the amount of displacement of the operation part 330 in the removal operation, resulting in improved operability. Further, the area of the surface (outer surface) where the user places his/her finger can be increased, resulting in improved operability.

The proximal end side engaging part 340 is a protrusion having a shape corresponding to the proximal end side engaging part 140 (recess) of the ultrasound probe body 100. When the puncture adapter 200 is attached to the ultrasound probe body 100, the proximal end side engaging part 340 is engaged with the proximal end side engaging part 140. The proximal end side engaging part 340 of this embodiment is formed at a position opposite to (below) the operation part 330 with respect to the connection position of the connector 320, and is configured to bend inward.

The proximal end side engaging part 340 is biased inwardly (in the direction of the ultrasound probe body 100) by the elastic force of the connector 320. With this biasing force, the proximal end side engaging part 340 engages the proximal end side engaging part 140 of the ultrasound probe body 100 (see FIG. 8A). That is, a pair of the proximal end side engaging parts 340 engages a pair of the proximal end side engaging parts 140 provided on both sides of the ultrasound probe body 100, and sandwiches the ultrasound probe body 100 from both sides. With this and the engagement of the distal end side engaging part 130 and the distal end side engaging part 430, the puncture adapter 200 is attached to the ultrasound probe body 100.

On the other hand, the puncture adapter 200 is removed from the ultrasound probe body 100 by bringing the pair of the operation parts 330 close to each other against the elastic force of the connector 320 as described above. If the pair of the operation parts 330 is displaced inward due to this removal operation, the connector 320 is deformed and thereby the proximal end side engaging part 340 is displaced outward. Accordingly, as illustrated in FIG. 8B, the engagement of the puncture adapter 200 (the proximal end side engaging part 340) to the ultrasound probe body 100 (the proximal end side engaging part 140) is released. Thus, the puncture adapter 200 can be removed from the ultrasound probe body 100.

### (Cover 400)

The cover 400 is provided with the guide groove 410, a displacement amount limiter 420, and the distal end side engaging part 430.

The displacement amount limiter 420 limits the amount of displacement of the operation parts 330 in a direction in which they come close to each other. The displacement amount limiter 420 of this embodiment is a protrusion that protrudes outward and is located in a position corresponding to the inside of the operation part 330 on both side surfaces of the cover 400. The height of the protrusion (protruding amount) is set arbitrarily. For example, the height of the protrusion is set such that, in the state where the ultrasound probe body 100 is fitted with the puncture adapter 200, the distance between the protrusion and the operation part 330 is slightly larger than the depth of the proximal end side engaging part 140 (recess). With this, it is possible to minimize the amount of displacement of the operation part 330 for releasing the engagement of the proximal end side engaging part 340 to the proximal end side engaging part 140. Thus, the connector 320 and the like are less likely to be broken.

The configuration of the displacement amount limiter of the embodiment is not limited to the above. For example, a protrusion may be provided on the inner surface of the operation part 330 to use the functional part that is brought in contact with a side surface of the cover 400 in response to the displacement of the operation part 330 as the displacement amount limiter.

The distal end side engaging part 430 is provided in the vicinity of the distal end on each of both side surfaces of the cover 400. The distal end side engaging part 430 is a protrusion having a shape corresponding to the distal end side engaging part 130 (recess) of the ultrasound probe body 100. When the puncture adapter 200 is attached to the ultrasound probe body 100, the distal end side engaging part 430 is engaged with the distal end side engaging part 130.

The ultrasound probe 1 of the embodiment includes the ultrasound probe body 100 and the puncture adapter 200. The ultrasound probe body 100 is inserted in a subject. The puncture adapter 200 holds a puncture needle and is attached to the ultrasound probe body 100. Incidentally, the puncture adapter 200 is an example of a puncture adapter according to an embodiment.

The puncture adapter 200 includes a main body, a pair of the connectors 320, a pair of the operation parts 330, and a pair of the proximal end side engaging parts 340. The main body has the first part 311 having the first width W1 and the second part 312 having the second width W2 that is narrower than the first width W1.

In the adapter body 300 of this embodiment, a portion that includes the puncture needle installation part 310, the first part 311, and the second part 312, excluding the connectors 320, the operation parts 330, and the proximal end side engaging parts 340, corresponds to the main body.

The operation parts 330 come close to each other in response to operation for removing the puncture adapter 200 from the ultrasound probe body 100.

One of the proximal end side engaging parts 340 is integrally formed with one of the operation parts 330, and is engageable with the ultrasound probe body 100. The other of the proximal end side engaging parts 340 is formed integrally with the other of the operation parts 330, and is engageable with the ultrasound probe body 100.

One of the connectors 320 connects one end in the width direction of the second part 312 and a position between one of the operation parts 330 and one of the proximal end side engaging parts 340. The other of the connectors 320 connects the other end in the width direction of the second part 312 and a position between the other of the operation parts 330 and the other of the proximal end side engaging parts 340.

While the puncture adapter 200 is being removed from the ultrasound probe body 100, the operation parts 330 come close to each other, and in response to this, the proximal end side engaging parts 340 separate from each other. Accordingly, the engagement of the proximal end side engaging parts 340 to the ultrasound probe body 100 is released, thus enabling the detachment of the puncture adapter 200.

The ultrasound probe 1 (the puncture adapter 200) is provided with the connector 320 on the side surface of the narrow second part 312 in the main body. Accordingly, the length of the connector 320 can be increased. That is, according to the embodiment, the length of the connector 320 can be secured on the main body side. Therefore, without increasing the width of the puncture adapter 200, in particular the width at the position of the pair of the operation parts 330, the required operating force can be reduced by lengthening the connector 320. Therefore, according to this embodiment, the puncture adapter can be easily removed from the ultrasound probe as in a small external shape.

The difference between the first width W1 and the second width W2 is set arbitrarily. If the difference is increased, the connector 320 can be longer, while the strength and rigidity of the second part 312 become relatively large with respect to the first part 311. On the other hand, if the difference is reduced, the strength and rigidity of the entire main body are improved; however, the connector 320 becomes shorter under a limiting condition on the external size of the puncture adapter 200. The difference between the first width W1 and the second width W2 can be set in consideration of these factors. As one embodiment, the second width W2 is set to be substantially 0.8 times the first width W1.

In the ultrasound probe 1 of this embodiment, each of the connectors 320 is formed thinner than the main body. Thereby, it is possible to reduce the operating force required for the removal operation.

In the ultrasound probe 1 of this embodiment, the chamfer 321 is formed in the connection part between each of the connectors 320 and the main body. The chamfer 321 can increase the strength of the connection part between the second part 312 and the connector 320.

The effects of the chamfer 321 are described in more detail. Since the connector 320 is connected to the narrow second part 312, the length of the connector 320 can be secured and thus the deformation easily occurs, resulting in a higher risk of breakage due to excessive deformation. In particular, the stress caused by the deformation concentrates on the connection part between the connector 320 and the second part 312. Accordingly, the connection part is likely to be broken. For this reason, in this embodiment, the chamfer 321 is provided to gradually reduce the thickness difference (to eliminate the step) between the second part 312 and the connector 320, thereby enabling the stress of deformation to be spread. Thus, it is possible to reduce the risk of breakage of the puncture adapter 200.

In the ultrasound probe 1 of this embodiment, the connector 320 has the ribs 322 along a direction that connects the second part 312 and a position between the operation part 330 and the proximal end side engaging part 340. The ribs 322 having an appropriate height enable the removal operation to be performed with a suitable operating force.

In the ultrasound probe 1 of this embodiment, the displacement amount limiters 420 are provided to limit the amount of displacement of the pair of the operation parts 330 in a direction in which they come close to each other. This makes it possible to limit the amount of deformation of the connector 320 and the like along with the displacement of the operation part 330. Thus, the connectors 320 and the like are less likely to be broken. In addition, it is possible to increase the life of the puncture adapter 200.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A puncture adapter configured to hold a puncture needle and be attached to an ultrasound probe that is configured to be inserted in a subject, the puncture adapter comprising:
a main body including a first part having a first width and a second part having a second width that is narrower than the first width;
a pair of operation parts configured to come close to each other in response to operation for removing the puncture adapter from the ultrasound probe;
a pair of engaging parts, each of which is integrally formed with either one of the operation parts, the engaging parts configured to be engageable with the ultrasound probe; and
a pair of connectors, one of which connects one end in a width direction of the second part and a position between one of the operation parts and one of the engaging parts, another connects another end in the width direction of the second part and a position between another of the operation parts and another of the engaging parts, wherein
when the operation parts come close to each other in response to the operation, the engaging parts separate from each other to release engagement.

2. The puncture adapter of claim 1, wherein the second width is substantially 0.8 times the first width.

3. The puncture adapter of claim 1, wherein each of the connectors is formed thinner than the main body.

4. The puncture adapter of claim 3, wherein a chamfer is formed in a connection part between each of the connectors and the second part.

5. The puncture adapter of claim 4, wherein the chamfer is a C-chamfer.

6. The puncture adapter of claim 1, wherein
one of the connectors has ribs along a direction that connects the second part and a position between one of the operation parts and one of the engaging parts, and
another of the connectors has ribs along a direction that connects the second part and a position between another of the operation parts and another of the engaging parts.

7. The puncture adapter of claim 1, further comprising a displacement amount limiter configured to limit an amount of displacement of each of the operation parts in a direction in which the operation parts come close to each other.

8. A puncture adapter configured to hold a puncture needle and be attached to an ultrasound probe that is configured to be inserted in a subject, the puncture adapter comprising:
a pair of operation parts configured to come close to each other in response to operation for removing the puncture adapter from the ultrasound probe; and
a pair of connectors, each provided to one of the operation parts and having a predetermined length, the connectors configured to be deformed in response to the operation.

9. An ultrasound probe, comprising:
an ultrasound probe body configured to be inserted in a subject; and
a puncture adapter configured to hold a puncture needle and be attached to the ultrasound probe body, the puncture adapter including
a main body including a first part having a first width and a second part having a second width that is narrower than the first width;
a pair of operation parts configured to come close to each other in response to operation for removing the puncture adapter from the ultrasound probe body;
a pair of engaging parts, each of which is integrally formed with either one of the operation parts, the engaging parts configured to be engageable with the ultrasound probe body; and
a pair of connectors, one of which connects one end in a width direction of the second part and a position between one of the operation parts and one of the engaging parts, another connects another end in the width direction of the second part and a position between another of the operation parts and another of the engaging parts, wherein
when the operation parts come close to each other in response to the operation, the engaging parts separate from each other to release engagement.

10. An ultrasound probe, comprising:
an ultrasound probe body configured to be inserted in a subject; and
a puncture adapter configured to hold a puncture needle and be attached to the ultrasound probe body, the puncture adapter including
a pair of operation parts configured to come close to each other in response to operation for removing the puncture adapter from the ultrasound probe body; and
a pair of connectors, each provided to one of the operation parts and having a predetermined length, the connectors configured to be deformed in response to the operation.

11. The puncture adapter of claim 2, wherein each of the connectors is formed thinner than the main body.
